(19) United States
(12) Patent Application Publication
(10) Pub. No.: US 2009/0171119 A1
(43) Pub. Date: Jul. 2, 2009

(54) PROCESS FOR PREPARING MENTHYLAMIDES

(75) Inventors: Andreas Job, Köln (DE); Uwe Westeppe, Remscheid (DE); Lars Rodefeld, Leverkusen (DE)

Correspondence Address: LANXESS CORPORATION, 111 RIDC PARK WEST DRIVE, PITTSBURGH, PA, 15275-1112, US

(73) Assignee: SALTIGO GMBH, Leverkusen (DE)

(21) Appl. No.: 12/279,200

(22) PCT Filed: Feb. 13, 2007
(86) PCT No.: PCT/EP2007/001217

§ 371 Date: Jan. 2, 2009

(30) Foreign Application Priority Data
Feb. 21, 2006 (DE) 10 2006 007 883.7

(51) Int. Cl. C07C 231/10

(52) U.S. Cl. 564/123

(57) ABSTRACT

An improved process for preparing menthylamides is provided by reacting menthyl halides with magnesium or lithium and subsequent reaction with an isocyanate, a heterocycle or a carbamoyl chloride.

The present invention relates to an improved process for preparing menthylamides.

Menthol is known for its physiological cooling effect on the skin and the mucous membranes of the mouth and is used widely as flavouring in foods, beverages, dental creams mouthwash and also as a component in a multitude of toiletries, ointments and lotions for topical application. This cooling effect is a physiological effect resulting from the direct effect of menthol on the nerve endings of the human body which are responsible for perceiving hot or cold, and is not based on the latent heat of evaporation. It is assumed that menthol has a directly stimulating effect on the cold receptors at the nerve endings which, for their part, stimulate the central nervous system.

However, the use of menthol is limited because of its strong peppermint-like odour and because of its relatively high volatility.

DE-A-24 136 39 and DE-A-22 052 55 described for the first time special menthylamides which likewise have a powerful physiological cooling effect on the skin and on the mucous membranes of the body, especially those of the nose, the mouth, the throat and the gastrointestinal tract. This cooling effect is in many cases much longer-lasting than that achieved with menthol, but without being accompanied by the strong odour. The compounds also have lower volatility than menthol and are used as so-called cooling agents in the food industry. Their economical production is thus of great interest.

DE-A-22 052 55 and DE-A-24 136 39 disclose a process for preparing menthylamides in which, starting from menthyl chloride, a Grignard reagent is firstly formed, which is reacted in a subsequent step with carbon dioxide to give the menthylcarboxylic acid.

This intermediate is then reacted with a chlorinating agent such as thionyl chloride to give the corresponding menthyl acid chloride, which reacts with a suitable mono- or disubstituted amine in a further stage in the presence of a hydrogen chloride acceptor such as sodium hydroxide to give the desired menthylamide. This procedure is disadvantageous in that, starting from menthyl chloride, it requires four chemical synthesis stages before reaching the target molecule.

On account of the widespread use of menthylamides and derivatives thereof in the food industry, there was thus a need for an economical process for preparing these menthylamides that is as technically simple as possible.

Surprisingly, it has been found that the reaction of a menthyl halide with magnesium or lithium and then with an isocyanate, a heterocycle or a carbamoyl chloride leads directly to the desired menthylamide. This process represents a considerable shortening of the synthesis route hitherto.

The invention provides a process for preparing compounds of the general formula (I)

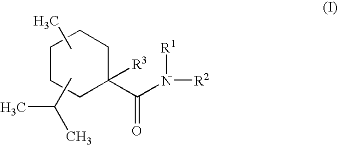

where
R¹ is C₁-C₃₀-alkyl, C₂-C₂₀-alkenyl, C₂-C₂₀-alkynyl, C₃-C₂₀-cycloalkyl, C₃-C₂₀-cycloalkenyl, C₃-C₂₀-cycloalkynyl, C₁-C₂₀-hydroxyalkyl, C₃-C₂₀-acyloxyalkyl, C₁-C₂₀-alkoxy, C₂-C₂₀-alkoxyalkyl, C₁-C₂₀-alkylthioalkyl, C₂-C₂₀-aminoalkyl, C₃-C₂₀-acylaminoalkyl, C₂-C₂₀-carboxyalkyl, a radical —CₙH₂ₙC(—O)R⁴ or a radical —CₙH₂ₙCOOR⁴, where —CₙH₂ₙ is a straight-chain or branched alkylene group in which n is an integer from 1 to 10 and R⁴ is hydrogen, C₁-C₁₀-alkyl, C₁-C₈-hydroxyalkyl, C₁-C₉-alkoxyalkyl or C₁-C₈-acyloxyalkyl,
R² can have the same meanings as R¹, may also be hydrogen or a radical —X—OH, where X is a straight-chain or branched C₁-C₂₀-alkylene group which is optionally interrupted by one or more heteroatoms, or
R¹ and R² together form an alkylene chain optionally interrupted by one or more heteroatoms which, taken together with the nitrogen atom to which R¹ and R² are bonded, forms a ring with a maximum of 10 members,
R³ is hydrogen, C₁-C₃₀-alkyl or C₆-C₂₄-arylalkyl,
   and the methyl radical and also the isopropyl radical can sit on all available carbon atoms of the cyclohexyl ring, but both radicals are not arranged on the same carbon atom,
   by firstly reacting a compound of the general formula (II)

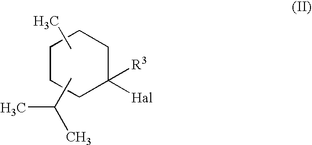

where
Hal is chlorine, bromine or iodine,
R³ has the meanings specified for the general formula (I)
   and the methyl radical and also the isopropyl radical can sit on all available carbon atoms of the cyclohexyl ring, but bot radicals are not arranged on the same carbon atom.
   with magnesium or lithium, and optionally elemental iodine or an alkyl halide, and then either with an isocyanate of the general formula (III),

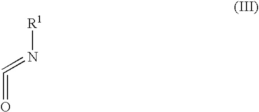

where R¹ has the meaning specified for the general formula (I) or with a heterocycle of the general formula (IV),

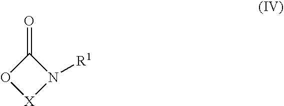

where R¹ and X have the meanings specified for the general formula (I),
or with a carbamoyl chloride of the general formula (V),

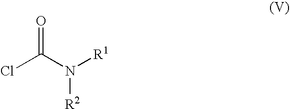

in which R¹ and R² have the meanings specified for the general formula (I).

In the compounds of the general formula (I) and (II), the methyl radical and also the isopropyl radical can sit on all available carbon atoms of the cyclohexyl ring, but both radicals are not arranged on the same carbon atom.

To make things easier, the compounds of the general formula (I) are also referred to as menthylamides.

Preference is given to the following compounds of the formula (II) which have a 2 isopropyl-5-methylcyclohexyl radical, 4-isopropyl-3-methylcyclohexyl radical or 2-isopropyl-4-methylcyclohexyl radical.

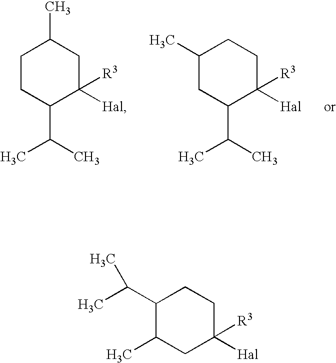

Their use leads to correspondingly substituted compounds of the general formula (I).

In the general formulae (I) and (II), R³ is hydrogen, C₁-C₃₀-alkyl or C₆-C₂₄-arylalkyl, preferably hydrogen, C₁-C₁₀-alkyl or C₆-C₁₁-arylalkyl, in particular methyl, ethyl, n-propyl, isopropyl, n-butyl, sec-butyl, tert-butyl, n-pentyl, 1-methylbutyl, 2-methylbutyl, 3-methylbutyl, neopentyl, 1-ethylpropyl, or benzyl.

Preparation of the starting material of the general formula (II) is possible by methods known to the person skilled in the art. Thus, for example, the preparation of the special menthyl chloride from menthol is described extensively in the literature, e.g. in J. Org, Chem. 2000, 65, 2337.

The reaction of the starting material of the general formula (II) to give the Grignard reagent can be carried out in various solvents. (see, for example, J. Org. Chem. 2000, 65, 2337 with regard to possible solvents for the Grignard reagent preparation). Suitable solvents are THF, dioxane, methyl-THF, cyclopentyl methyl ether or mixtures of the aforementioned solvents with aromatic solvents such as toluene or xylene.

Isocyanates which can be used are isocyanates of the general formula (III) which have the following general, preferred or particularly preferred meanings for R. These give compounds of the general formula (I) which have the corresponding general, preferred or particularly preferred meaning of R¹ and in which R² is hydrogen.

In the general formulae (I) and (III), R¹ can be
C₁-C₃₀-alkyl, C₂-C₂₀-alkenyl, C₂-C₂₀-alkynyl, C₃-C₂₀-cycloalkyl, C₃-C₁₀-cycloalkenyl, C₃-C₂₀-cycloalkynyl, C₁-C₂₀-hydroxyalkyl, C₃-C₂₀-acyloxyalkyl, C₁-C₂₀-alkoxy, C₂-C₂₀-alkoxyalkyl, C₂-C₂₀-alkylthioalkyl, C₂-C₂₀-aminoalkyl, C₃-C₂₀-acylaminoalkyl, C₂-C₂₀-carboxyalkyl, a radical —CₙH₂ₙC(═O)R⁴ or a radical —CₙH₂ₙCOOR⁴, where —CₙH₂ₙ is a straight-chain or branched alkylene group in which n is an integer from 1 to 10 and R⁴ is hydrogen, C₁-C₁₀-alkyl, C₁-C₈-hydroxyalkyl, C₁-C₈-alkoxyalkyl or C₁-C₈-acyloxyalkyl.

All of the aforementioned meanings for the radical R¹ which have alkyl, alkenyl, alkynyl radicals may in each case be branched or unbranched.

Preferably, R¹ in the general formulae (I) and (III) is
C₁-C₁₄-alkyl, C₂-C₁₀-alkenyl, C₂-C₁₀-alkynyl, C₅-C₁₀-cycloalkyl, C₅-C₁₀-cycloalkenyl, C₅-C₁₀-cycloalkynyl, C₁-C₁₀-hydroxyalkyl, C₃-C₁₀-acyloxyalkyl, C₁-C₁₀-alkoxy, C₂-C₁₀-alkoxyalkyl, C₂-C₁₀-alkylthioalkyl, C₂-C₁₀-aminoalkyl, C₃-C₁₀-acylaminoalkyl, C₂-C₁₀-carboxyalkyl, a radical —CₙH₂ₙC(═O)R⁴ or a radical —CₙH₂ₙCOOR⁴, where —CₙH₂ₙ is a straight-chain or branched alkylene group in which n is an integer from 1 to 6 and R⁴ is hydrogen, C₁-C₁₀-alkyl, C₁-C₈-hydroxyalkyl, C₁-C₈-alkoxyalkyl or C₁-C₈-acyloxyalkyl.

Particularly preferably, R¹ in the general formulae (I) and (III) is a straight-chain or branched C₁-C₁₀-alkyl radical, such as methyl, ethyl, n-propyl, isopropyl, n-butyl, sec-butyl, tert-butyl, n-pentyl, 1-methylbutyl, 2-methylbutyl, 3-methylbutyl, neopentyl, 1-ethylpropyl, cyclohexyl, cyclopentyl, n-hexyl, 1,1-dimethylpropyl, 1,2-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-hexyl, n-heptyl, n-octyl, n-nonyl or n-decyl.

Alternatively to the isocyanates of the general formula (III), it is also possible to use heterocycles of the general formula (IV), where R¹ can assume all general, preferred or particularly preferred meanings which R¹ can have in the general formula (II), and X is a straight-chain or branched C₁-C₂₀-alkylene group, preferably a straight-chain or branched C₁-C₈-alkylene group, particularly preferably a methylene group, ethylene group, n-propylene group, n-butylene group or n-pentylene group, where these general, preferred or particularly preferred alkylene groups are optionally interrupted by one or more heteroatoms, preferably oxygen or sulphur.

If, instead of the isocyanate of the general formula (III), a heterocycle of the general formula (IV) is used, then hydroxy-functionalized amides are obtained.

Alternatively to the isocyanates of the general formula (III) and the heterocycles of the general formula (IV), it is also possible to use carbamoyl chlorides of the general formula (V),

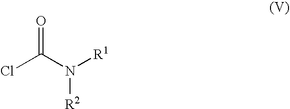

where R¹ and R² can have all of the general, preferred and particularly preferred meanings specified above for the general formula (I).

The compounds of the general formula (I) have both geometric isomers and also optical isomers and, depending on the starting materials and methods used in their preparation, may be isomerically pure, i.e. consist of just one geometric or optical isomer, or else be isomer mixtures, both in the geometric and in the optical sense. The basic structure of the cyclohexyl ring is a chair-form molecule which may be present in the cis form or trans form in relation to the methyl and isopropyl radical. Using the example of 2-isopropyl-5-methyleyclohexylcarboxamide, this means that the substitution of the cyclohexyl ring leads, through the carboxamide group —CONHR¹ in the 1 position, to four configuration or geometric isomers depending on whether the substitution is axial or equatorial in the cis or trans isomer. Furthermore, optical isomers arise for each of the aforementioned geometric isomers. The general formula (I) is therefore intended to encompass all possible stereoisomers as well as all possible diastereomers and enantiomers.

Usually, the process according to the invention is carried out by initially introducing magnesium or lithium under inert gas and admixing with a solvent. Elemental iodine or an alkyl halide, such as, for example, ethyl bromide, 1,2-dichloroethane or 1,2-dibromoethane, preferably ethyl bromide, is then optionally added. The addition of elemental iodine or an alkyl halide has proven useful primarily when using magnesium. The temperature here is kept in a range from 20° C. to 80° C. and adjusted according to the solvent used. The compound of the general formula (II) is then added. It has proven useful to dissolve this compound of the general formula (II) in the same solvent as that initially introduced. Usually, the molar ratio of magnesium or lithium to the compound of the general formula (II) is (0.9-1.6): 1, preferably (1.0-1.4):1. The addition takes place in a manner such that the temperature of the reaction mixture fluctuates within a range from 20° C. to 80° C. Here too, an adjustment to the solvent used can be made. The reaction mixture is then usually after stirred for a certain period of time, cooled to a temperature of from 20° C. to 50° C. and filtered off or decanted off.

The reaction with the further reagent in the form of the isocyanate of the general formula (III), of the heterocycle of the general formula (IV) or of the carbamoyl chloride of the general formula (V) then takes place. These compounds can either be used as they are, i.e. without a diluent, or else diluted in an organic solvent.

One variant of the procedure consists in adding the further reagent to the reaction mixture which is obtained in the reaction of the compound of the general formula (II) with magnesium or lithium. However, it is also possible to reverse the order of addition, i.e. to add the reaction mixture from the reaction of the compound of the general formula (II) with magnesium or lithium to the further reagent of the formula (III), (IV) or (V).

Usually, the molar ratio of the compound of the general formulae (III), (IV) or (V) to the compound of the general formula (II) is (0.95-1.4):1, preferably (1.0-1.2):1. It is also possible, prior to adding these reagents, to partially distil off the solvent used for the reaction with magnesium or lithium. In this case, the reagent to be added is then used advantageously in dissolved form in an organic solvent, for example toluene or xylene. When the reaction is complete, any low-boiling solvent originally used for the reaction with magnesium or lithium that is still present can be further distilled off and optionally returned.

When the reaction is complete, the reaction mixture is admixed with aqueous acid and the mixture is worked-up, for example by phase separation or extraction with an organic solvent. The desired compound of the general formula (I) is removed via the organic phases, dried and isolated.

The abovementioned steps of the reaction and also for the work-up can not only be carried out discontinuously, but also continuously.

If a continuous operation is used, then the solution obtained after reacting the compound of the general formula (II) with magnesium or lithium is advantageously transferred to the next reaction container via an overflow device, thus separating off the magnesium or lithium excess.

### EXAMPLES

### Example 1

Magnesium turnings (0.87 g) initially introduced under nitrogen were initially introduced into a heat-dried flask and admixed with 7.6 ml of THE. At 50° C., about 92 mg of ethyl bromide were added to start the reaction. Then, a solution of 5 g of menthyl chloride in 10.2 ml of THF was added to the mixture at 55° C. over 3.5 h. The mixture was then further stirred for 1.3 hours at 70° C. After cooling the mixture to 22° C., it was filtered and transferred to a further flask. 3.05 g of butyl isocyanate were added to the filtered reaction solution, which was further stirred for one hour. With cooling and stirring, this mixture was added to 127 g of 2% strength hydrochloric acid. The mixture was extracted with 3×20 ml of dichloromethane. The combined organic phases were dried over magnesium sulphate and concentrated by evaporation.

This gave 5.70 g of a pale yellow oil which, according to gas chromatographic analysis, comprised 53% product. This corresponds to a yield of 45% of theory.

### Example 2

Magnesium turnings (0.87 g) initially introduced under nitrogen were initially introduced into a heat-dried flask and admixed with 7.6 ml of TV. At 50° C., about 92 mg of ethyl bromide were added to start the reaction. Then, a solution of 5 g of menthyl chloride in 10.2 ml of TEE was added to the mixture at 55° C. over 3.5 h. The mixture was then further stirred for 1.3 hours at 70° C. After cooling the mixture to 22° C., it was filtered and transferred to a further flask. 2.23 g of ethyl isocyanate were added to the filtered reaction solution, which was further stirred for one hour. With cooling and stirring, this mixture was added to 127 g of 2% strength hydrochloric acid. The mixture was extracted with 3×20 ml of dichloromethane. The combined organic phases were dried over magnesium sulphate and concentrated by evaporation.

This gave 5.30 g of a pale yellow oil which, according to gas chromatographic analysis, comprised 43% product. This corresponds to a yield of 39% of theory.

1. Process for preparing compounds of the general formula (I) 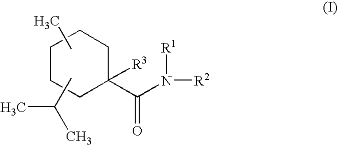
where
R¹ is C₁-C₃₀-alkyl, C₂-C₂₀-alkenyl, C₂-C₂₀-alkynyl, C₃-C₂₀-cycloalkyl, C₃-C₂₀-cycloalkenyl, C₃-C₂₀-cycloalkynyl, C₁-C₂₀-hydroxyalkyl, C₃-C₂₀-acyloxyalkyl, C₁-C₂₀-alkoxy, C₂-C₂₀-alkoxyalkyl, C₂-C₂₀-alkylthioalkyl, C₂-C₂₀-aminoalkyl, C₃-C₂₀-acylaminoalkyl, C₂-C₂₀-carboxyalkyl, a radical —CₙH₂ₙC(═O)R⁴ or a radical —CₙH₂ₙCOOR⁴ where —CₙH₂ₙ is a straight-chain or branched alkylene group in which n is an integer from 1 to 10 and R⁴ is hydrogen, C₁-C₁₀-alkyl, C₁-C₈-hydroxyalkyl, C₁-C₈-alkoxyalkyl or C₁-C₈-acyloxyalkyl,
R² can have the same meanings as R¹, may also be hydrogen or a radical —X—OH, where X is a straight-chain or branched C₁-C₂₀-alkylene group which is optionally interrupted by one or more heteroatoms, or
R¹ and R² together form an alkylene chain optionally interrupted by one or more heteroatoms which, taken together with the nitrogen atom to which R¹ and R² are bonded, forms a ring with a maximum of 10 members,
R³ is hydrogen, C₁-C₃₀-alkyl or C₆-C₂₄-arylalkyl,
and the methyl radical and also the isopropyl radical can sit on all available carbon atoms of the cyclohexyl ring, but both radicals are not arranged on the same carbon atom,
by firstly reacting a compound of the general formula (II) 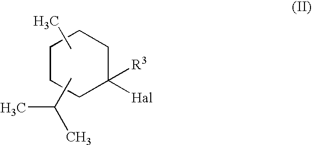
where
Hal is chlorine, bromine or iodine,
R³ has the meanings specified for the general formula (I)
and the methyl radical and also the isopropyl radical can sit on all available carbon atoms of the cyclohexyl ring, but both radicals are not arranged on the same carbon atom,
with magnesium or lithium, and optionally elemental iodine or an alkyl halide, and then
either with an isocyanate of the general formula (III), 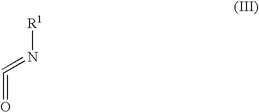
where R¹ has the meaning specified for the general formula (I)
or with a heterocycle of the general formula (IV), 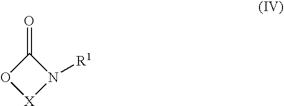
where R¹ and X have the meanings specified for the general formula (I),
or with a carbamoyl chloride of the general formula (V), 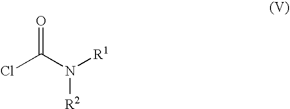
in which R¹ and R² have the meanings specified for the general formula (I).

2. Process according to claim 1, where the compounds of the general formula (II) used are either

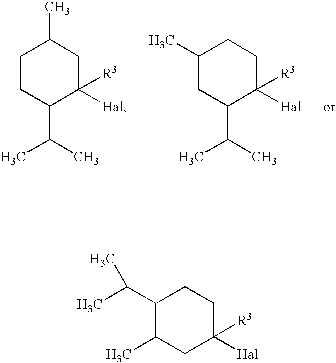

3. Process according to claim 1 or 2, where R³ in the general formulae (I) and (II) is hydrogen, C₁-C₁₀-alkyl or C₆-C₁₁-arylalkyl, preferably methyl, ethyl, n-propyl, isopropyl, n-butyl, sec-butyl, tert-butyl, n-pentyl, 1-methylbutyl, 2-methylbutyl, 3-methylbutyl, neopentyl, 1-ethylpropyl or benzyl

4. Process according to one or more of claims 1 to 3, where isocyanates of the general formula (III) are used in which R¹ is C₁-C₁₄-alkyl, C₂-C₁₀-alkenyl, C₂-C₁₀-alkynyl, C₅-C₁₀-cycloalkyl, C₅-C₁₀-cycloalkenyl, C₅-C₁₀-cycloalkynyl, C₁-C₁₀-hydroxyalkyl, C₃-C₁₀-acyloxyalkyl, C₁-C₁₀-alkoxy, C₂-C₁₀-alkoxyalkyl, C₂-C₁₀-alkylthioalkyl, C₂-C₁₀-aminoalkyl, C₃-C₁₀-acylaminoalkyl, C₂-C₁₀-carboxyalkyl, a radical —CₙH₂ₙC(═)OR⁴ or a radical —CₙH₂ₙCOOR⁴, where —CₙH₂ₙ is a straight-chain or branched alkylene group in which n is an integer from 1 to 6 and R⁴ is hydrogen, C₁-C₁₀-alkyl, C₁-C₈-hydroxyalkyl, C₁-C₈-alkoxyalkyl or C₁-C₈-acyloxyalkyl.

5. Process according to one or more of claims 1 to 4, where isocyanates of the general formula (III) are used in which R¹ is methyl, ethyl, n-propyl, isopropyl, n-butyl, sec-butyl, tert-butyl, n-pentyl, 1-methylbutyl, 2-methylbutyl, 3-methylbutyl, neopentyl, 1-ethylpropyl, cyclohexyl, cyclopentyl, n-hexyl, 1,1-dimethylpropyl, 1,2-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-hexyl, n-heptyl, n-octyl, n-nonyl or n-decyl.

6. Process according to one or more of claims 1 to 3, where heterocycles of the general formula (IV) are used where R¹ can assume all of the meanings specified for R¹ in claims 1 to 5 and X is a straight-chain or branched C₁-C₈-alkylene group, particularly preferably methylene, ethylene, n-propylene, n-butylene, n-pentylene, where this alkylene group is optionally interrupted by one or more heteroatoms, preferably oxygen or sulphur.

7. Process according to one or more of claims 1 to 3, where carbamoyl chlorides of the general formula (V) are used where R¹ and R² can assume all of the meanings specified for R¹ and R² in claims 1 to 5.

8. Process according to one or more of claims 1 to 7, characterized in that magnesium or lithium is initially introduced under inert gas and admixed with a solvent, then optionally elemental iodine or an alkyl halide, preferably ethyl bromide, 1,2-dichloroethane or 1,2-dibromoethane, is added, where the temperature is kept in a range from 20° C. to 80° C., then the compound of the general formula (II) is added, and is reacted with the isocyanate of the general formula (III), the heterocycle of the general formula (IV) or the carbamoyl chloride of the general formula (V).
